(19) United States
(12) Patent Application Publication
(10) Pub. No.: US 2026/0294963 A1
(43) Pub. Date: Oct. 1, 2026

(54) MAGNESIUM THREONATE COMPOSITIONS AND USE FOR IMPROVING SLEEP

(71) Applicant: Threotech LLC, City Of Industry, CA (US)

(72) Inventors: Jennifer Gu, Diamond Bar, CA (US); Mike Weiser, Erie, CO (US); Edward Lee, Diamond Bar, CA (US); Guosong Liu, Walnut Creek, CA (US)

(21) Appl. No.: 19/091,203

(22) Filed: Mar. 26, 2025

(51) Int. Cl. A61K 33/06; A61P 25/20

(52) U.S. Cl. CPC A61K 33/06; A61P 25/20

(57) ABSTRACT

The present disclosure relates to the use of magnesium salts, such as magnesium threonate, for improving sleep in human subjects in need thereof.

### BACKGROUND

Recent polling suggests that 62% of adults globally report poor sleep quality. Magnesium has been implicated to benefit sleep, although the clinical evidence varies based on the magnesium source used, and there is limited randomized placebo-controlled clinical trial evidence to support the benefits of magnesium in sleep, potentially due to the poor brain bioavailability of the magnesium compounds used in those studies. Accordingly, there is a need for magnesium sources with improved brain bioavailability for use in improving sleep and cognition.

### SUMMARY OF THE INVENTION

In certain aspects, provided herein are methods for improving sleep in a human subject in need thereof, comprising administering to the subject an effective amount of magnesium or a salt thereof, wherein the effective amount is from about 1 mg to about 300 mg based on the weight of magnesium.

In certain aspects, provided herein are methods for improving sleep in a subject in need thereof, comprising administering to the subject an effective amount of magnesium threonate.

In certain aspects, provided herein are methods for improving sleep quality in a human subject in need thereof, comprising administering to the subject an effective amount of magnesium threonate.

In certain aspects, provided herein are methods for improving mood in a human subject in need thereof, comprising administering to the subject an effective amount of magnesium threonate.

In certain aspects, provided herein are methods for improving energy and alertness in a human subject in need thereof, comprising administering to the subject in need an effective amount of magnesium threonate.

In certain aspects, provided herein are methods for improving daily productivity in a human subject in need thereof, wherein the method comprising administering to the subject in need an effective amount of magnesium threonate.

In certain aspects, provided herein are methods for improving morning freshness in a human subject in need thereof, wherein the method comprising administering to the subject in need an effective amount of magnesium threonate.

### BRIEF DESCRIPTION OF THE DRAWINGS

FIG. 1 shows a diagram of participant flow through recruitment, consenting, intervention and outcome measurement process for the clinical study of the use of magnesium threonate for improving sleep described herein.

FIG. 2A is a plot demonstrating that Magnesium L-threonate (black symbols) showed significant (p<0.05) improvements over placebo (white symbols) for Leeds Sleep Questionnaire (LSEQ) subcategory behavior following awakening. Data shown are mean and standard deviation (n=38 per group).

FIG. 2B is a plot demonstrating that Magnesium L-threonate (black symbols) showed improvements over placebo (white symbols) for Restorative Sleep Questionnaire (RSQ) subcategories grouchy. Data shown are mean and standard deviation (n=38 per group).

FIG. 2C is a plot demonstrating that Magnesium L-threonate (black symbols) showed improvements over placebo (white symbols) for Restorative Sleep Questionnaire (RSQ) subcategories in a good mood. Data shown are mean and standard deviation (n=38 per group).

FIG. 2D is a plot demonstrating that Magnesium L-threonate (black symbols) showed over placebo (white symbols) for Restorative Sleep Questionnaire (RSQ) subcategories in mental alertness. Data shown are mean and standard deviation (n=38 per group).

### DETAILED DESCRIPTION OF THE INVENTION

Globally, 62% of adults report poor sleep quality according to a commercial poll. Recent sleep quality literature suggests insomnia within a Scandinavian population of over 21,000 respondents ranges as much as 8.5%-23.6%, depending on the diagnostic criteria. Other populations report similar ranges, for example 3.9%-22.1% in the US and 4.7%-22.1% in Hong Kong, again depending on diagnostic criteria. In older people, the prevalence of sleep disorders ranges from 9.1% to 69%. Types of sleep disturbances include insomnia, sleep disordered breathing, circadian rhythm sleep disorder, and others. Irrespective of the source and criteria, clearly these substantial prevalences of sleep disturbance are concerning since poor sleep quality is associated with increased risk for physical, cognitive, and mental health issues such as obesity, diabetes, high blood pressure, coronary heart disease, depression, dementia, and stroke. Furthermore, poor sleep quality commonly leads to negative activity states such as poor mood, greater anxiety, and reduced energy and productivity.

Magnesium, a key cofactor in numerous enzymatic reactions, is proposed to enhance neuroplasticity and synaptic function. This would be expected to enhance the brain's capacity to adapt to emotional challenges and mitigate stress responses. Furthermore, magnesium may regulate neurotransmitters such as glutamate and gamma-aminobutyric acid (GABA). Regulating GABA, an inhibitory neurotransmitter that promotes relaxation and reduces neural excitability, may have a calming effect that benefits sleep. Additionally, magnesium is important for the synthesis of N-acetyltransferase, which converts 5-hydroxytryptamine (5-HT) into N-acetyl-5-hydroxytryptamine, which can then be converted to melatonin. Increased melatonin secretion leads to improved sleep onset. It was reported that plasma magnesium levels are lower in sleep deprived people (e.g., sleep less than 7 h per night). Magnesium has previously been associated with improved sleep length and quality. Observational studies found magnesium helps in maintaining a normal circadian rhythm, reducing daytime sleepiness, and improving sleep quality. However, there is limited randomized placebo-controlled clinical trial evidence to support the benefits of magnesium in sleep, potentially due to the poor brain bioavailability of the magnesium compounds used in those studies, and therefore it is uncertain based on the existing literature how to effectively utilize magnesium supplements to improve sleep.

Magnesium L-threonate is a magnesium salt with superior brain bioavailability. Magnesium L-threonate has been shown in animal studies to effectively deliver magnesium through the blood-brain barrier into the neuron cells leading to increased neural plasticity, improved memory and cognition, and reduced anxiety and stress. These benefits have also been demonstrated in humans. Magnesium in the form of a salt with L-threonate, an endogenous compound found in cerebrospinal fluid, appears to be a viable form for this brain delivery of magnesium.

The present disclosure addresses the potential that Magnesium L-threonate would also have benefits for sleep. The objectives were to investigate the impact of Magnesium L-threonate supplementation on sleep parameters, as well as on mood, daytime energy, and other related measurements. With this randomized placebo-controlled double-blind trial, the effectiveness of magnesium supplementation on sleep quality and daytime activity is examined, using Magnesium L-threonate in healthy adults with non-clinical insomnia symptoms (e.g., self-assessed sleep problems). Participants underwent Magnesium L-threonate supplementation for 21 days, with sleep and daytime mood and activity outcomes assessed through standardized psychometric questionnaire accompanied by objective Oura ring measurements, with the aim of discerning the effects of Magnesium L-threonate on sleep, daily functioning, and emotional states.

Primary outcomes measures were sleep quality measured weekly with the Insomnia Severity Index, Leeds Sleep Evaluation Questionnaire and Restorative Sleep Questionnaire, and the Oura ring nightly. Secondary outcome measures were a weekly mood questionnaire, daily activity measured by Oura ring, daily diary and study perceived effectiveness using a post-study questionnaire at day 21.

Eighty 35-55-year-olds with self-assessed sleep problems participated in a randomized, double-blind, placebo-controlled, parallel-arm study, taking 1 g/day of Magnesium L-threonate, or placebo for 21 days. Sleep and daily behaviors were measured subjectively using standardized questionnaires including the Insomnia Severity Index, Leeds Sleep Evaluation Questionnaire, and Restorative Sleep Questionnaire, and objectively using an Oura ring. The Profile of Mood States questionnaire and a daily diary were used to evaluate mood, energy and productivity, and record any safety concerns.

The Magnesium L-threonate group maintained good sleep quality and daytime functioning, while placebo declined. From objective Oura ring measurements, Magnesium L-threonate significantly (p<0.05) improved vs placebo deep sleep score, REM sleep score, light sleep time, and activity and readiness parameters activity score, activity daily movement score, readiness score, readiness activity balance, and readiness sleep balance. From subjective questionnaires, Magnesium L-threonate significantly (p<0.05) improved vs placebo behavior upon awakening, energy and daytime productivity, grouchiness, mood and mental alertness. Magnesium L-threonate was safe and well tolerated.

This showed Magnesium L-threonate improved sleep quality, especially deep/REM sleep stages, improved mood, energy, alertness, and daily activity and productivity. These are consistent with how Magnesium L-threonate works in neuron cells and animal models, suggesting broader positive impacts on overall brain health.

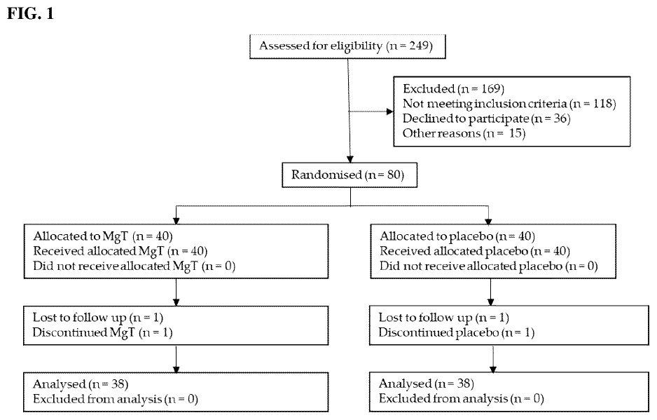

As detailed in FIG. 1, from 249 people assessed for eligibility, 169 were excluded from the study. Those not meeting inclusion criteria were 118 with 36 declined to further participate, and 15 withdrew for other reasons. Of the remaining 80 who enrolled and consented, 40 were randomized into each treatment group, Magnesium L-threonate and placebo. Of the Magnesium L-threonate group, all 40 received allocation of Magnesium L-threonate, one was lost to follow up, and one discontinued for unknown reasons. All remaining 38 were analyzed. Of the placebo group, all 40 received allocation of placebo, one was lost to follow up, and one discontinued for unknown reasons. All remaining 38 were analyzed. All data was collected from completing participants, including Oura ring data.

Participants (Table 1) had an average age of 45.49 years (SD=6.00; age range=35-55 years). All participants exhibited nonclinical insomnia symptoms, as indicated by their scores on the Insomnia Severity Index (ISI), with a mean score of 12.46 (SD=3.37), according to the assessment by Bastien and coworkers.

TABLE 1
Baseline sociodemographic and clinical characteristics.
Data shows mean and standard deviation.

| | | | P value |
|---|---|---|---|
| | | | (Paired |
| Parameter | MgT (n = 40) | Placebo (n = 40) | Sample T-test) |
| Age | 46.99 ± 6.08 | 43.63 ± 5.68 | 0.012 |
| BMI | 24.51 ± 3.84 | 25.19 ± 4.19 | 0.489 |
| Sex | n = 7 (17.5%) | n = 10 (25%) males | |
| | males | n = 30 (75%) females | |
| | n = 33 (82.5%) | | |
| | females | | |
| Ethnicity | n = 37 (92.5%) | n = 34 (85%) White/ | |
| | White/Caucasian | Caucasian | |
| | n = 1 (2.5%) | n = 3 (7.5%) White/ | |
| | Hispanic | Caucasian & Asian/Asian | |
| | n = 1 (2.5%) | American | |
| | Hispanic | n = 2 (5%) Asian/Asian | |
| | n = 1 (2.5%) | American | |
| | Black & Hispanic | n = 1 2.5%) Hispanic | |
| | n = 1 (2.5%) | | |
| | Asian/Asian | | |
| | American | | |
| Insomnia | 12.46 ± 3.37 | 12.41 ± 3.71 | 0.977 |
| Severity | | | |
| Index | | | |

For the ISI, as demonstrated in Table 2, there was a significance improvement for condition (p<0.001) and time (p<0.01). Magnesium L-threonate group significantly improved total ISI score from baseline to day 7, 14 and 21, while placebo also significantly improved total ISI score from baseline to day 7, 14 and 21, but there was no statistically significant interaction (condition×time) (p=0.39). Interestingly, despite the high effect in the placebo group, only the Magnesium L-threonate group appeared to continue to improve by days 14 and 21, while placebo group plateaus after the first week. There is a significant difference between the changes from week 1 to week 2 (p=0.023) and week 1 to week 3 (p=0.0001) of the ISI between Magnesium L-threonate and placebo groups.

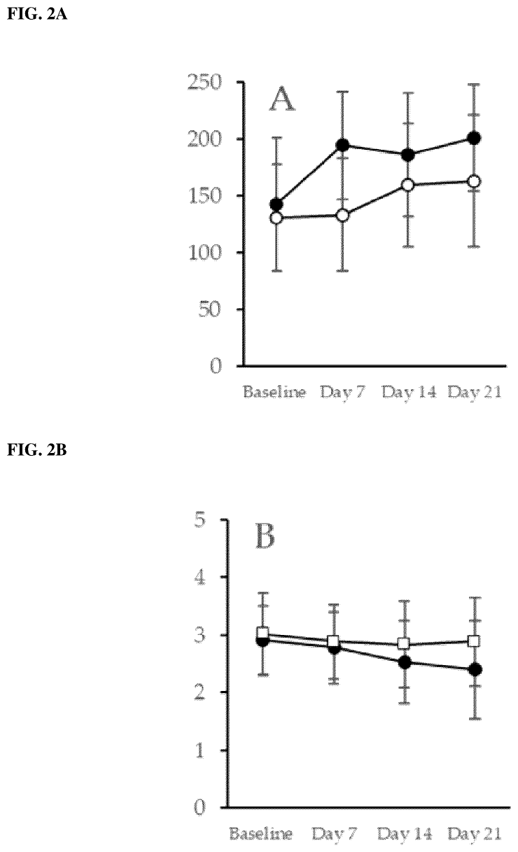

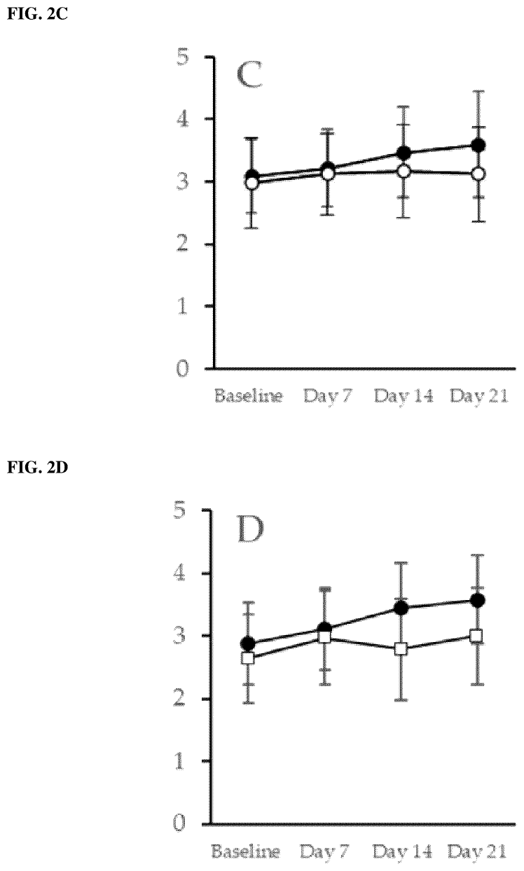

For the LSEQ, as demonstrated in Table 2 and FIG. 2A, the behavior following awakening subscale showed a significant interaction (condition×time) (p=0.004). However, the awake following sleep; sleep quality (i.e., calmer sleep with less wakeful periods); and getting to sleep (i.e., ease and time to fall asleep) subscales each showed significant effects for condition (p<0.001) and time (p<0.01) but no significant interaction (awake following sleep, p=0.65; sleep quality, p=0.81; and getting to sleep, p=0.25), due to a strong response from the placebo group. For the behavior following awakening subscale showing a significant interaction, a post hoc analysis (T-test) was conducted comparing Magnesium L-threonate to placebo. The Magnesium L-threonate group showed a significant improvement on day 7 (p<0.001), day 14 (p=0.023), and day 21 (p=0.003) over the placebo.

These data showed that participants all felt sleep quality improvements, but the Magnesium L-threonate group demonstrated significantly better results over the placebo in the behavior upon awakening subcategory, which includes alertness, coordination and balance following awakening.

For the RSQ, as demonstrated in Table 2 and FIG. 2B-D, the grouchy (FIG. 2B), in a good mood (FIG. 2C) and mentally alert (FIG. 2D) subscales showed a significant condition×time interaction (grouchy, p=0.044; good mood, p=0.044; and mentally alert, p=0.003). Significant main effects for condition, (p<0.001) and time, (p<0.01) for all three subscales were also demonstrated. For the values showing a significant interaction, post hoc analyses (T-tests) was conducted. For grouchy, the Magnesium L-threonate group showed significant reduction at day 14 (p=0.084) and day 21 (p=0.020) as compared to placebo. For in a good mood, Magnesium L-threonate improved significantly at day 14 (p=0.083) and day 21 (p=0.012) versus placebo. For mentally alert, Magnesium L-threonate again significantly improved at day 7 (p=0.051), day 14 (p<0.001), and day 21 (p<0.001). As demonstrated in Table 1, there were also significant main effects for condition (p<0.001) and time (p<0.010), but not interaction (p>0.05) for the subscales tired, sleepy, rested, refreshed and restored, ready to start the day and energetic, due to high responses from the placebo group.

From the daily diary assessments, as shown in Table 2, the Magnesium L-threonate group showed significantly greater improvements to energy and day-time productivity than the placebo group, both with significant condition (p<0.001), time (p<0.010) and condition×time interaction (p=0.004).

TABLE 2
Insomnia Severity Index (ISI), Leeds Sleep Evaluation Questionnaire (LSEQ), Restorative Sleep
Questionnaire (RSQ) and daily diary results (mean and standard deviation, n = 38 per group).

| | | | | | | | | | | | Interaction |
|---|---|---|---|---|---|---|---|---|---|---|---|
| | | | | | | | | | | | (Condition × |
| | [symbol] | | | | Placebo | | | | Condition | Time | Time) |
| | Baseline | Day 7 | Day 14 | Day 21 | Baseline | Day 7 | Day 14 | Day 21 | P value | P value | P value [symbol] |
| Insomnia Severity | 12.32 | [symbol] | 0.16 | [symbol] | 12.57 | 9.22 | 9.34 | 9.39 | <0.001 | <0.01 | 0.390 |
| Index | (4.25) | (3.09) | (4.49) | (4.79) | ( [symbol] ) | (4.15) | (4.43) | (4.04) | | | |
| LSEQ Behavior | 142.37 | [symbol] | [symbol] | [symbol] | 130.71 | [symbol] | 159.67 | 163.25 | <0.001 | <0.010 | 0.004 |
| following | ( [symbol] ) | (47.02) | (54.07) | (46.83) | (46.76) | (49.21) | ( [symbol] ) | ( [symbol] ) | | | |
| awakening | | | | | | | | | | | |
| LSEQ Awake | 94.42 | 124.11 | 121.94 | 124.75 | 83.57 | [symbol] | 105.62 | 100.91 | <0.001 | <0.001 | [symbol] |
| following sleep | ( [symbol] ) | (33.09) | (31.79) | (38.48) | (86.68) | (34.66) | (24.64) | (36.39) | | | |
| LSEQ Quality | 73.36 | 107.46 | 114.45 | [symbol] | 77.16 | 105.1 | [symbol] | 109.9 | <0.001 | <0.001 | 0.810 |
| of sleep | (40.65) | ( [symbol] ) | (27.75) | (34.77) | (33.21) | (27.70) | (39.89) | (82.98) | | | |
| LSEQ [symbol] | 159.11 | [symbol] | 164.57 | 183.46 | [symbol] | 177.36 | 173.46 | 176.62 | <0.001 | <0.001 | 0.250 |
| to sleep | (43.00) | (25.47) | (39.95) | (37.90) | (51.49) | ( [symbol] ) | (51.63) | (40.32) | | | |
| RSQ Total Score | 24.16 | [symbol] | 26.13 | [symbol] | 22.23 | 23.41 | 23.25 | 23.45 | <0.001 | <0.010 | 0.134 |
| | (0.10) | ( [symbol] ) | (3.18) | (3.57) | (2.67) | ( [symbol] ) | ( [symbol] ) | ( [symbol] ) | | | |
| RSQ Grouchy (R) | 2.92 | [symbol] | 2.53 | 2.4 | 3.02 | 2.88 | 2.83 | 2.88 | <0.001 | <0.010 | 0.044 |
| | (0.59) | ( [symbol] ) | (0.72) | ( [symbol] ) | (0.72) | (0.65) | (0.75) | (0.76) | | | |
| RSQ In a good mood | 3.08 | 3.22 | 3.47 | 3.60 | [symbol] | 3.12 | 3.17 | 3.12 | <0.001 | <0.010 | 0.044 |
| | (0.59) | (0.62) | (0.72) | ( [symbol] ) | (0.72) | (0.65) | (0.75) | (0.76) | | | |
| RSQ Mentally alert | 2.87 | 3.11 | 3.45 | [symbol] | 2.64 | 2.97 | 2.79 | [symbol] | <0.001 | <0.010 | [symbol] |
| | ( [symbol] ) | (0.65) | (0.71) | (0.71) | (0.7) | (0.75) | ( [symbol] ) | (0.77) | | | |
| RSQ Sleepy (R) | 2.76 | 3.65 | 3.66 | 3.8 | [symbol] | [symbol] | 3.55 | 3.60 | <0.001 | <0.010 | [symbol] |
| | (0.79) | (0.94) | (0.87) | ( [symbol] ) | (0.95) | ( [symbol] ) | ( [symbol] ) | (0.90) | | | |
| RSQ Tired (R) | 2.49 | [symbol] | 3.36 | 3.49 | 2.62 | 3.24 | 3.19 | 3.41 | <0.001 | <0.010 | 0.238 |
| | (0.47) | (0.65) | (0.84) | ( [symbol] ) | (0.94) | ( [symbol] ) | (0.76) | (0.91) | | | |
| RSQ Rested | 2.11 | 2.71 | 2.95 | 3.02 | 1.99 | 2.46 | 2.47 | 2.5 | <0.001 | <0.010 | 0.144 |
| | ( [symbol] ) | (0.93) | (1.09) | (1.03) | (0.72) | (0.92) | (0.95) | ( [symbol] ) | | | |
| RSQ Refreshed and | [symbol] | 2.74 | 2.78 | 2.75 | 1.79 | 2.36 | 2.31 | 2.47 | <0.001 | <0.010 | 0.702 |
| restored | (0.77) | (0.92) | (0.96) | (0.02) | (0.78) | (0.94) | (0.84) | (0.95) | | | |
| RSQ Ready to start | 2.61 | 3.05 | 3.39 | 3.3 | 2.27 | 2.63 | 2.66 | 2.68 | <0.001 | <0.010 | 0.267 |
| the day | (0.09) | ( [symbol] ) | (0.78) | (0.95) | (0.09) | (1.05) | (0.99) | (0.99) | | | |
| RSQ Energetic | 2.58 | 3.04 | 3.14 | 3.18 | 2.04 | 2.39 | 2.43 | 2.5 | <0.001 | <0.010 | 0.829 |
| | (0.53) | (0.52) | (0.62) | (0.8) | (0.76) | (1.05) | (0.97) | (1.08) | | | |
| Daily diary Energy | [symbol] | 6.41 | 6.78 | 6.92 | 6.54 | 6.45 | 6.73 | 6.71 | <0.001 | <0.010 | 0.004 |
| (1-10 low-high) | ( [symbol] ) | ( [symbol] ) | (1.67) | (1.63) | (1.54) | (1.64) | (1.55) | (1.79) | | | |
| Daily diary | 6.29 | 6.54 | 6.99 | 6.99 | 6.77 | [symbol] | 6.81 | 6.86 | <0.001 | <0.010 | 0.004 |
| Productivity (1-10 | (2.05) | ( [symbol] ) | (1.75) | (1.79) | (1.85) | ( [symbol] ) | (1.62) | (1.77) | | | |
| low-high) | | | | | | | | | | | |

(R): reverse scoring (lower number indicates improvement). [symbol]
p < 0.05 indicates significance between groups. [symbol]
indicates data missing or illegible when filed

For the POMS short form, as shown in Table 3, POMS subscales anger and esteem showed significant condition×time interactions (both p=0.020). Significant main effects for condition (both p<0.001) and time (both p<0.001) were also recorded. Post hoc analyses (T-tests) showed that Magnesium L-threonate was significantly improved at day 21 (p=0.026) for the esteem subscale as compared to the placebo.

TABLE 3
Profile of Mood States (POMS) questionnaire results (mean and standard deviation, n = 38 per group).

| | | | | | | | | | | | Interaction |
|---|---|---|---|---|---|---|---|---|---|---|---|
| | [symbol] | | | | Placebo | | | | Condition | Time | (Condition × Time) |
| | Baseline | Day 7 | Day 14 | Day 21 | Baseline | Day 7 | Day 14 | Day 21 | P value | P value | P value [symbol] |
| POMS: | [symbol] | [symbol] | [symbol] | 153.32 | 159.46 | 150.26 | 148.91 | [symbol] | <0.001 | <0.001 | 0.480 |
| Total | (14.22) | (12.57) | (11.44) | (12.99) | (13.32) | ( [symbol] ) | (10.73) | (10.12) | | | |
| Anger | 6.14 | [symbol] | 4.96 | [symbol] | 7.14 | [symbol] | 4.03 | 3.98 | <0.001 | <0.001 | 0.020 |
| | (3.64) | (3.04) | (3.03) | ( [symbol] ) | (4.33) | (3.7) | ( [symbol] ) | (3.12) | | | |
| Esteem | 14.69 | 15.94 | 16.96 | 17.84 | 14.76 | 15.59 | 15.75 | [symbol] | <0.001 | <0.001 | 0.020 |
| | (2.57) | (3.25) | (2.70) | ( [symbol] ) | (3.60) | (3.40) | (2.98) | ( [symbol] ) | | | |
| Tension | 10.42 | 7.55 | 6.70 | 6.35 | [symbol] | 7.26 | 6.25 | 5.55 | <0.001 | <0.001 | 0.900 |
| | (4.32) | (3.40) | ( [symbol] ) | ( [symbol] ) | (4.42) | (3.56) | (3.44) | (2.66) | | | |
| Fatigue | 9.5 | 7.74 | 6.59 | 6.22 | 9.07 | 7.27 | 6.60 | 7.01 | <0.001 | <0.001 | 0.220 |
| | (3.35) | (4.07) | (3.75) | (4.16) | (3.89) | ( [symbol] ) | (3.45) | (3.32) | | | |
| Depression | 6.17 | [symbol] | 3.77 | 2.90 | 5.76 | 3.91 | 2.80 | 2.73 | <0.001 | <0.001 | 0.250 |
| | (4.29) | (4.94) | (3.17) | (2.44) | (4.48) | (3.29) | (2.65) | (2.85) | | | |
| Vigor | [symbol] | 10.08 | 11.20 | 11.22 | 7.10 | 7.74 | 8.74 | 8.66 | <0.001 | <0.001 | 0.790 |
| | (2.78) | (2.93) | (2.91) | ( [symbol] ) | (3.84) | (4.14) | (4.67) | (4.52) | | | |
| Confusion | 6.55 | 5.06 | 4.26 | [symbol] | [symbol] | 4.29 | 3.61 | 2.71 | <0.001 | <0.001 | 0.550 |
| | (3.30) | (2.70) | ( [symbol] ) | (2.77) | (3.15) | (2.71) | (2.22) | (2.22) | | | |

[symbol]

p < 0.05 indicates significance between groups. [symbol]
indicates data missing or illegible when filed

Using objective Oura ring measurements, as demonstrated in Table 4, the sleep parameters that showed a significant interaction (condition×time) included deep sleep score (p<0.001), REM sleep score (p=0.020), light sleep time (p=0.006). For the activity and readiness parameters that showed a significant interaction (condition×time) included activity score (p=0.010), activity daily movement score (p<0.001), readiness score (p=0.010), readiness activity balance (p=0.003), and readiness sleep balance (p<0.001). For the values showing a significant interaction, a post hoc analyses (T-tests) was conducted. Magnesium L-threonate was significantly better than the placebo for deep sleep score at day 14 (p=0.040). Comparing the activity score, Magnesium L-threonate was significantly different than placebo at day 7 (p=0.044) and day 21 (p=0.028). Comparing activity daily movement score, Magnesium L-threonate approached significance at day 7 (p=0.059) and was significantly improved at day 14 (p=0.035) and day 21 (p=0.044) over placebo. Comparing readiness activity balance, Magnesium L-threonate approached significance for day 7 (p=0.086), as compared to placebo.

TABLE 4
Oura Ring sleep results (mean and standard deviation, n = 38 per group)

| | | | | | | | | | | | Interaction |
|---|---|---|---|---|---|---|---|---|---|---|---|
| | | | | | | | | | | | (Condition × |
| | [symbol] | | | | Placebo | | | | Condition | Time | Time) |
| Sleep parameters | Baseline | Day 7 | Day 14 | Day 21 | Baseline | Day 7 | Day 14 | Day 21 | P value | P value | P value [symbol] |
| Time awake | [symbol] | 54.71 | [symbol] | 57.39 | 51.75 | 53.29 | 54.34 | 55.82 | <0.001 | 0.110 | 0.950 |
| (Minutes) | ( [symbol] ) | (26.44) | (27.39) | (28.21) | ( [symbol] ) | ( [symbol] ) | (24.43) | (26.75) | | | |
| Sleep Latency | 9.07 | 9.64 | 10.39 | [symbol] | 10.07 | 11.06 | 9.86 | 9.99 | <0.001 | 0.280 | 0.050 |
| (Minutes) | (6.00) | (6.57) | (6.20) | (6.34) | (6.28) | (6.77) | (6.44) | (6.05) | | | |
| Sleep duration | 476.52 | 485.41 | 479.57 | [symbol] | 497.29 | 497.85 | [symbol] | 498.17 | <0.001 | [symbol] | 0.580 |
| (Minutes) | (66.2) | ( [symbol] ) | (68.5) | ( [symbol] ) | (66.31) | (65.39) | ( [symbol] ) | (74.04) | | | |
| Light Sleep | 218.89 | [symbol] | 223.70 | 218.18 | 227.53 | 222.53 | 229.79 | 236.21 | <0.001 | 0.530 | 0.006 |
| (Minutes) | (46.63) | ( [symbol] ) | (44.69) | (44.72) | (57.2) | (50.87) | (53.77) | (50.59) | | | |
| REM Sleep | 97.75 | 99.86 | 97.10 | 95.76 | 107.37 | [symbol] | [symbol] | 104.10 | <0.001 | 0.570 | 0.190 |
| (minutes) | (33.51) | (34.09) | (33.7) | (32.65) | (37.55) | (37.05) | (37.91) | (38.77) | | | |
| Deep Sleep | 96.3 | 92.73 | 94.75 | 97.75 | 100.52 | 99.55 | 99.53 | 93.65 | <0.001 | 0.490 | 0.070 |
| (minutes) | (33.01) | ( [symbol] ) | (33.52) | (36.2) | (34.7) | (35.85) | (41.09) | (37.12) | | | |
| Total Sleep | 417.88 | 425.13 | 420.57 | [symbol] | 439.54 | 438.94 | 437.5 | 440.43 | <0.001 | 0.550 | 0.590 |
| (Minutes) | (52.31) | (62.06) | (56.68) | (58.31) | (60.9) | (62.22) | ( [symbol] ) | ( [symbol] ) | | | |
| Sleep Efficiency | 58.24 | 83.73 | 88.17 | [symbol] | [symbol] | 89.19 | 89.03 | 58.59 | <0.001 | 0.010 | 0.590 |
| | (5.2) | ( [symbol] ) | (4.94) | (5.82) | (4.12) | (4.43) | (4.83) | (4.95) | | | |
| Sleep Disturbance | 75.44 | 75.78 | 75.24 | 74.62 | 76.77 | [symbol] | 75.50 | 74.97 | <0.001 | 0.050 | 0.760 |
| Score | (8.64) | (8.84) | ( [symbol] ) | ( [symbol] ) | (8.67) | ( [symbol] ) | (10.40) | (9.95) | | | |
| Sleep Efficiency | 89.41 | [symbol] | 59.07 | [symbol] | 92.14 | 91.07 | 90.54 | 89.4 | <0.001 | <0.001 | 0.210 |
| Score | (9.85) | (9.61) | (9.9) | (15.15) | (6.99) | (8.63) | (8.19) | (9.69) | | | |
| Latency Sleep Score | 73.92 | 79.65 | 81.46 | 79.6 | 80.18 | 80.81 | 79.73 | 80.49 | <0.001 | 0.540 | 0.100 |
| | (10.83) | (10.86) | (10.98) | (10.31) | (10.11) | (10.62) | (10.37) | (10.36) | | | |
| REM Sleep Score | 84.65 | [symbol] | 86.42 | 84.82 | 88.91 | 85.96 | 84.55 | 85.61 | <0.001 | 0.190 | 0.020 |
| | (17.37) | (14.77) | (15.26) | ( [symbol] ) | ( [symbol] ) | (15.15) | (17.87) | (16.52) | | | |
| Deep Sleep Score | 93.84 | 92.65 | [symbol] | 94.2 | 93.58 | 94.05 | [symbol] | 90.7 | <0.001 | <0.001 | <0.001 |
| | (8.33) | (10.31) | ( [symbol] ) | (0.54) | (9.18) | ( [symbol] ) | (12.73) | (12.99) | | | |
| Total Sleep Score | [symbol] | [symbol] | 80.72 | [symbol] | 84.26 | 83.5 | 82.58 | 83.64 | <0.001 | [symbol] | 0.420 |
| | (12.43) | ( [symbol] ) | (12.58) | (12.76) | ( [symbol] ) | (12.59) | (13.12) | (13.6) | | | |
| Overall Sleep Score | [symbol] | 83.4 | [symbol] | [symbol] | 84.6 | 84.19 | 82.96 | [symbol] | <0.001 | 0.070 | 0.130 |
| | (7.25) | (7.3) | (7.05) | ( [symbol] ) | (6.07) | (6.94) | ( [symbol] ) | (7.64) | | | |

Activity and readiness parameters

| | | | | | | | | | | | |
|---|---|---|---|---|---|---|---|---|---|---|---|
| Activity Score | [symbol] | 85.19 | [symbol] | [symbol] | 86.17 | 78.61 | [symbol] | [symbol] | <0.001 | <0.001 | 0.010 |
| | ( [symbol] ) | (11.34) | (10.58) | (9.02) | (11.16) | (16.18) | (15.67) | (16.77) | | | |
| Activity Met Daily | 81.33 | 81.90 | [symbol] | [symbol] | [symbol] | 84.74 | 84.44 | [symbol] | <0.001 | 0.880 | 0.810 |
| Movement Goals | (13.94) | [symbol] | (13.34) | (13.49) | (12.94) | (12.30) | (13.1) | (12.52) | | | |
| Activity Daily | 79.72 | 79.59 | [symbol] | [symbol] | 76.52 | 65.62 | 64.74 | 64.6 | <0.001 | [symbol] | <0.001 |
| Movement Score | (20.26) | [symbol] | (24.55) | (22.93) | (25.47) | ( [symbol] ) | (38.45) | (39.62) | | | |
| Readiness Score | 79.75 | [symbol] | 80.01 | 80.16 | 82.21 | 81.52 | [symbol] | 79.95 | <0.001 | <0.001 | 0.010 |
| | (7.56) | (6.86) | (6.41) | (7.24) | (6.69) | (6.97) | (6.27) | (7.02) | | | |
| Readiness Activity | 82.69 | [symbol] | 82.85 | 84.75 | 83.69 | 85.89 | 85.54 | 84.9 | <0.001 | 0.007 | [symbol] |
| Balance | (7.25) | (9.91) | (8.77) | (7.17) | (13.19) | (7.73) | (8.11) | (8.97) | | | |
| Readiness Sleep | [symbol] | 81.97 | 80.17 | [symbol] | 88.24 | [symbol] | 81.09 | 82.74 | <0.001 | 0.710 | <0.001 |
| Balance | (9.51) | ( [symbol] ) | (11.06) | (12.2) | (7.52) | (11.06) | (10.07) | (11.46) | | | |

[symbol]

p < 0.05 indicates significance between groups. [symbol]
indicates data missing or illegible when filed

Overall, the Oura ring data shows Magnesium L-threonate maintained good sleep quality and daytime functioning, significantly better than the placebo group.

For the post-study questionnaires, as shown in Table 5, at the end of the intervention period, significantly (p<0.05) more of the Magnesium L-threonate group as compared to the placebo group answered YES to the following questions: feeling their sleep was more refreshing/restoring, feeling more rested upon awakening, feeling their physical health had improved, and feeling their mental health had improved, feeling their concentration during the daytime improved, feeling they had more energy during the daytime, and feeling more alert during the daytime.

TABLE 5
Post-study self-report assessments (percentage answering “yes”, n = 38 per group)

| | | | Chi- |
|---|---|---|---|
| Question | MgT | Placebo | square |
| Has the quality of your sleep improved? | 57% | 42% | χ² = 0.15 |
| Is your sleep more refreshing/restoring? | 57% | 29% | χ² = 0.01^{a} |
| Do you feel more rested upon awakening? | 62% | 29% | χ² = 0.004^{a} |
| Do you feel that your physical health has | 49% | 24% | χ² = 0.022^{a} |
| improved? | | | |
| Do you feel that your mental health has improved? | 59% | 29% | χ² = 0.007^{a} |
| Do you feel calmer? | 57% | 42% | χ² = 0.15 |
| Do you feel that your memory during the daytime | 41% | 24% | χ² = 0.09 |
| improved? | | | |
| Do you feel that your concentration during the | 46% | 24% | χ² = 0.04^{a} |
| daytime improved? | | | |
| Do you feel like you have more energy during the | 51% | 29% | χ² = 0.04^{a} |
| daytime? | | | |
| Do you feel more alert during the daytime? | 59% | 26% | χ² = 0.004^{a} |
| Taking the supplement had a positive effect on my | 46% | 26% | χ² = 0.06 |
| perceived stress and anxiety. | | | |
| Taking the supplement had a positive effect on my | 33% | 23% | χ² = 0.24 |
| mood. | | | |

^{a}p < 0.05 indicates significance between groups.

### Adverse Event Recording

Participant comments from daily diaries were assessed for possible adverse events. Four AEs were recorded from the Magnesium L-threonate group, and 13 from the placebo group. None were regarded as “probable” or higher by MedDRA severity classifications. For the Magnesium L-threonate group AEs recorded using MedDRA terminology were nocturnal awakening (1), cramps legs (2) and sunken eyes (1). From placebo group AEs recorded were nocturnal awakening (2), sleepiness (6), feeling anxious (1), restlessness (3), pulse increased (1). Magnesium L-threonate appeared to be safe and well-tolerated.

Magnesium L-threonate has previously been clinically demonstrated to improve cognitive scores reflecting individual's executive function, working memory, episodic memory, and attention by six weeks. Similarly, it has previously been demonstrated to significantly improve memory, associational learning, figure recognition, recall and character face association. Additionally, it was shown to reduce self-reported stress and anxiety after twelve weeks consumption while also reducing clinically administered fear scores after six- and twelve-weeks consumption. Furthermore, it was reported in an eight-week open label study with 15 dementia patients to improve overall cognitive functioning and in a twelve-week open-label study with ADHD participants to improve self-reported ADHD symptoms, executive function, and visual scanning measures. These are consistent with animal data. Magnesium has always been implicated to benefit sleep, but clinical evidence is not always conclusive, most likely because most of the magnesium compounds used in the studies are not brain bioavailable. Magnesium L-threonate is able to cross the blood brain barrier and increase magnesium levels in the brain and neuron cells. Thus, it is of great interest to examine the effect of Magnesium L-threonate on sleep. The effects of 1.0 g daily Magnesium L-threonate (about 75 mg of bioavailable elemental magnesium) intake over three weeks were reported. The results of this study demonstrated that supplementation with Magnesium L-threonate alone is safe, well-tolerated and resulted in statistically significant improvements in sleep quality, daytime activity, and mood outcomes, as compared to placebo.

Magnesium L-threonate improves sleep outcomes. For self-reported sleep quality, the Magnesium L-threonate-consuming group had significantly improved behavior following awakening LSEQ subscale, mental alertness, good mood and were less grouchy in the RSQ measurements compared to the placebo group. Nonsignificant improvements (i.e., trending in the right direction) were evidenced for the Magnesium L-threonate compared to the placebo for most other LSEQ and RSQ parameters; they did not reach statistical significance, apparently due to the strong placebo effects that is often associated with subjective measures. However, it is interesting that most of the improvements observed in the placebo happened in the first week of product intake, and the effects plateaued afterwards, while in the Magnesium L-threonate group, the effects continued to improve for the rest of the study. Other magnesium sleep studies also showed strong placebo responses.

With this study, measures recorded from a wearable device (Oura ring) were more objective and thus statistically more definitive than the participant-assessed sleep outcomes. Magnesium L-threonate significantly outperformed the placebo for deep sleep score (p<0.001), activity score (p=0.010), activity daily movement score (p<0.001), readiness score (p=0.010), and readiness activity balance (p=0.003), light sleep (p=0.006), REM sleep score (p=0.020) and readiness sleep balance (p<0.001). Deep sleep is the most restorative and rejuvenating sleep stage. During deep sleep, breathing is slow, heartbeat is regular, and muscles are relaxed; and it is the period where the body heals itself (e.g., replaces cells, builds muscle tissue, and heals wounds). Deep sleep encompasses between 0 and 35% of total sleep, and the average adult spends 15-20% of their total sleep time in deep sleep. In terms of cognition, deep sleep contributes to thinking, creativity, and memory consolidation and restoration, specifically procedural and declarative memory. This deep sleep stage allows the brain to rest and recover, which assists with improving cognitive function, concentration, and alertness. Plus during deep sleep the brain processes and integrates new information, which can improve memory retention and learning. It's found that Magnesium L-threonate supplementation improved deep sleep score, which provides an explanation for previous findings that Magnesium L-threonate improved cognitive abilities compared to placebo. It is plausible that some of the cognitive enhancing effects of Magnesium L-threonate may, in part, be due to its ability to improve deep sleep, although the causal/effect connection still needs to be investigated. Relatedly, magnesium improves synapse configuration associated with learning and new memory formation, those functions perhaps occurring during deep sleep.

Insufficient sleep is linked to reduced energy, productivity, and physical activity as well as increased feeling of fatigue. With Magnesium L-threonate supplementation, individuals demonstrated an improved sleep quality, and an improved daytime mental clarity and physical activity. The Magnesium L-threonate results support previous research findings of a bidirectional relationship between improved sleep quality and increased daytime activity. It is also interesting to notice that Magnesium L-threonate slightly increased sleep latency as compared to the placebo (p<0.05) in this study, suggesting that Magnesium L-threonate's benefits on sleep is more on the improved deep sleep quality and refreshed awakening, improved daily activity and mental alertness, rather than helping people to fall sleep faster.

Finally, in vitro work shows increasing magnesium by one third from 0.8 mM to 1.2 mM, within biologically plausible concentrations, improves neuronal cell energy in terms of increased mitochondrial function by 68% within 4 h, doubling neuronal ATP production. It occurs to us that improved neuronal energy in response to Magnesium L-threonate may be a source of the improved daytime functioning observed by the participants in this study.

Magnesium L-threonate improves mood. It is reported that 1 g daily Magnesium L-threonate consumption improved good mood (Table 2), reduced grouchiness (Table 2) and reduced anger (Table 3). This highlights the role of Magnesium L-threonate in improving behavior (and mood) upon awakening. Magnesium consumption has been linked to improved mood previously, albeit weakly, perhaps due to the lower brain bioavailability of other types of magnesium. It is found that the mood improvements reported here to be statistically significant as compared to the placebo. This observation is consistent with the improved quality of sleep, suggesting that the benefits of Magnesium L-threonate is multi-functional, which can be expected from the multi-functional benefits of magnesium in overall brain health.

Magnesium L-threonate starts working within 14 days. It is observed that, the effects of Magnesium L-threonate intake reached significance as compared to the placebo by 7-14 days into this study, namely LSEQ behavior following awakening and Oura ring activity score (days 7-21); RSQ mentally alert, Oura ring deep sleep and activity daily movement (days 14-21). This is the first human study which has demonstrated the response time of Magnesium L-threonate as within 7-14 days (Tables 1-3).

Magnesium L-threonate outperforms considerable placebo effects. From the self-assessed outcomes, It is reported here, it was evident that almost all participants reported their sleep quality and their behavior improved over the three weeks, irrespective of treatment. This highlighted a particularly strong placebo effect with these self-assessed scoring questionnaires. Placebo effects are often observed in various clinical studies using these subjective questionnaires. These effects often exhibit temporal dynamics that can be influenced by factors such as the duration of the intervention, the nature of the outcome measure, and individual expectations.

In terms of duration, placebo effects on mood and anxiety often manifests as short-term improvements in self-reported measures. However, the sustainability of these effects over longer periods is often limited. The transient nature of placebo effects in non-diagnosed participants underscores the importance of distinguishing between short-term and lasting changes. A large part of this difference is, obviously, actual treatment efficacy versus participant expectation. Expectation plays a significant role in the temporal aspects of the placebo effect. When participants believe they are receiving an active intervention, their expectations often lead to rapid improvements. These effects are often particularly pronounced in the early stages of an intervention but diminish as participants become more aware, thus lacking long-term effect. This placebo effect is also observed in this study. In most of the measurements using questionnaires, both Magnesium L-threonate group and placebo group showed a statistical significance over the baseline (as evidenced by the significant p values of condition and time separately) but failed to reach significance when compared between Magnesium L-threonate and placebo (as evidenced by the p value of the interaction). In these measurements, the Magnesium L-threonate group appeared to continue to improve after the first week, while placebo group showed most effect in the first week and plateaued afterwards.

Neurobiological mechanisms do underline placebo effects, even in non-diagnosed participants. In terms of mood and anxiety, neuro-imaging studies have shown that placebo interventions can lead to changes in brain activity patterns associated with emotional processing and mood regulation, as well as motor control, endocrine and immune effects. These mechanisms can contribute to the temporal dynamics of placebo responses in mood and anxiety. These effects may be driven by neurotransmitter activity or brain activation patterns: Short-term placebo effects might involve the release of neurotransmitters like endorphins, dopamine, and serotonin, which are associated with sleep and mood regulation. These neurotransmitters can lead to immediate improvements, but sustained changes may require more comprehensive neural adaptations.

As mentioned previously, Magnesium L-threonate has been shown in animals to result in neural adaptations which improved memory outcomes, and similar outcome benefits in terms of memory and cognition (as well as mood) have been observed in humans. It may be safe to conclude that such adaptations have occurred here.

Collectively, these data showing improved sleep quality and daytime functioning from daily consumption of magnesium L-threonate suggest they may play a role in clinical practice as an adjunct therapy for healthy individuals experiencing sleeplessness or the consequences thereof. Both magnesium and threonic acid ligand are endogenously present in the body) and possess a good safety profile. Together with the demonstrated benefits in sleep, cognition, mood and many other areas of brain health, magnesium L threonate would be the choice of brain magnesium in clinical practices. Research into the role magnesium L-threonate plays in areas such as restoring or ameliorating memory deficit, neuroinflammation and pain, ADHD, stress and anxiety have occurred and are ongoing.

### METHODS OF THE DISCLOSURE

In certain aspects, provided herein are methods for improving sleep in a human subject in need thereof, comprising administering to the subject an effective amount of magnesium or a salt thereof, wherein the effective amount is from about 1 mg to about 300 mg based on the weight of magnesium.

In certain preferred embodiments, the effective amount of the magnesium or a salt thereof is from about 1 mg to about 100 mg based on the weight of magnesium. In certain preferred embodiments, the effective amount of the magnesium or salt thereof is from about 72 mg to about 83 mg based on the weight of magnesium. In further preferred embodiments, the effective amount is about 81 mg based on the weight of magnesium. In yet further preferred embodiments, the method comprises administering a magnesium salt, wherein the effective amount is an amount which is sufficient to deliver from about 72 mg to about 83 mg of magnesium to the subject per gram of the magnesium salt. In still further preferred embodiments, the method comprises administering a magnesium salt, wherein the effective amount is an amount which is sufficient to deliver about 75 mg of magnesium to the subject per gram of the magnesium salt. In certain embodiments, the magnesium salt is magnesium threonate. In further embodiments, the effective amount of magnesium threonate is from about 100 mg to about 5 g. In further embodiments, the effective amount of magnesium threonate is from about 100 mg to about 1.3 g.

In further aspects, provided herein are methods for improving sleep in a subject in need thereof, comprising administering to the subject an effective amount of magnesium threonate.

In yet further aspects, provided herein are methods for improving sleep quality in a human subject in need thereof, comprising administering to the subject an effective amount of magnesium threonate.

In certain embodiments, the sleep is deep sleep. In certain embodiments, the sleep is Rapid Eye Movement (REM) sleep.

In still further aspects, provided herein are methods for improving mood in a human subject in need thereof, comprising administering to the subject an effective amount of magnesium threonate.

In certain aspects, provided herein are methods for improving energy and alertness in a human subject in need thereof, comprising administering to the subject in need an effective amount of magnesium threonate.

In further aspects, provided herein are methods for improving daily productivity in a human subject in need thereof, wherein the method comprising administering to the subject in need an effective amount of magnesium threonate.

In yet further aspects, provided herein are methods for improving morning freshness in a human subject in need thereof, wherein the method comprising administering to the subject in need an effective amount of magnesium threonate.

In certain embodiments, the effective amount of magnesium threonate is from about 50 mg to about 5 g. In further embodiments, the effective amount of magnesium threonate is from about 50 mg to about 4 g. In yet further embodiments, the effective amount of magnesium threonate is from about 50 mg to about 3 g. In still further embodiments, the effective amount of magnesium threonate is from about 50 mg to about 2 g. In certain embodiments, the effective amount of magnesium threonate is from about 500 mg to about 5 g. In further embodiments, the effective amount of magnesium threonate is from about 500 mg to about 4 g. In yet further embodiments, the effective amount of magnesium threonate is from about 500 mg to about 3 g. In still further embodiments, the effective amount of magnesium threonate is from about 500 mg to about 2 g. In certain preferred embodiments, the effective amount of magnesium threonate is from about 1 g to about 2 g.

In certain embodiments, the effective amount of magnesium threonate is about 1 g. In further preferred embodiments, the effective amount of magnesium threonate is about 1.1 g. In yet further embodiments, the effective amount of magnesium threonate is about 1.2 g. In still further embodiments, the effective amount of magnesium threonate is about 1.3 g. In certain embodiments, the effective amount of magnesium threonate is about 1.4 g. In further embodiments, the effective amount of magnesium threonate is about 1.5 g. In yet further embodiments, the effective amount of magnesium threonate is about 1.6 g. In still further embodiments, the effective amount of magnesium threonate is about 1.7 g. In certain embodiments, the effective amount of magnesium threonate is about 1.8 g. In further embodiments, the effective amount of magnesium threonate is about 1.9 g. In yet further embodiments, the effective amount of magnesium threonate is about 2.0 g.

In certain embodiments, the effective amount of magnesium threonate is an amount which is sufficient to deliver from about 1 mg to about 500 mg of magnesium to the subject. In further embodiments, the effective amount of magnesium threonate is an amount which is sufficient to deliver from about 1 mg to about 400 mg of magnesium to the subject. In yet further embodiments, the effective amount of magnesium threonate is an amount which is sufficient to deliver from about 1 mg to about 300 mg of magnesium to the subject. In still further embodiments, the effective amount of magnesium threonate is an amount which is sufficient to deliver from about 1 mg to about 200 mg of magnesium to the subject. In certain embodiments, the effective amount of magnesium threonate is an amount which is sufficient to deliver from about 1 mg to about 100 mg of magnesium to the subject. In further preferred embodiments, the effective amount of magnesium threonate is an amount which is sufficient to deliver from about 72 mg to about 83 mg of magnesium to the subject.

In certain embodiments, the effective amount of magnesium threonate is an amount which is sufficient to deliver about 1 mg of magnesium to the subject. In further embodiments, the effective amount of magnesium threonate is an amount which is sufficient to deliver about 10 mg of magnesium to the subject. In yet further embodiments, the effective amount of magnesium threonate is an amount which is sufficient to deliver about 20 mg of magnesium to the subject. In still further embodiments, the effective amount of magnesium threonate is an amount which is sufficient to deliver about 30 mg of magnesium to the subject. In certain embodiments, the effective amount of magnesium threonate is an amount which is sufficient to deliver about 40 mg of magnesium to the subject. In further embodiments, the effective amount of magnesium threonate is an amount which is sufficient to deliver about 50 mg of magnesium to the subject. In yet further embodiments, the effective amount of magnesium threonate is an amount which is sufficient to deliver about 60 mg of magnesium to the subject. In still further embodiments, the effective amount of magnesium threonate is an amount which is sufficient to deliver about 70 mg of magnesium to the subject.

In certain preferred embodiments, the effective amount of magnesium threonate is an amount which is sufficient to deliver about 75 mg of magnesium to the subject. In further embodiments, the effective amount of magnesium threonate is an amount which is sufficient to deliver about 80 mg of magnesium to the subject. In yet further embodiments, the effective amount of magnesium threonate is an amount which is sufficient to deliver about 83 mg of magnesium to the subject. In still further embodiments, the effective amount of magnesium threonate is an amount which is sufficient to deliver about 90 mg of magnesium to the subject. In certain embodiments, the effective amount of magnesium threonate is an amount which is sufficient to deliver about 100 mg of magnesium to the subject.

In certain embodiments, the magnesium threonate is magnesium L-threonate.

In certain embodiments, the subject is a healthy subject. In further embodiments, the subject does not have cognitive impairment. In yet further embodiments, the subject is an adult.

In certain embodiments, the magnesium, the salt thereof, or the magnesium threonate is administered orally. In further embodiments, the magnesium, the salt thereof, or the magnesium threonate is administered to the subject once per day. In yet further embodiments, the magnesium, the salt thereof, or the magnesium threonate is administered to the subject twice per day.

In certain embodiments, the magnesium, the salt thereof, or the magnesium threonate is administered to the subject about 30 minutes before sleep. In further embodiments, the magnesium, the salt thereof, or the magnesium threonate is administered to the subject about 1 hour before sleep. In yet further embodiments, the magnesium, the salt thereof, or the magnesium threonate is administered to the subject about 1.5 hours before sleep. In certain preferred embodiments, the magnesium, the salt thereof, or the magnesium threonate is administered to the subject about 2 hours before sleep.

In certain embodiments, the magnesium, the salt thereof, or the magnesium threonate is administered to the subject about 2.5 hours before sleep. In further embodiments, the magnesium, the salt thereof, or the magnesium threonate is administered to the subject about 3 hours before sleep. In yet further embodiments, the magnesium, the salt thereof, or the magnesium threonate is administered to the subject about 3.5 hours before sleep. In still further embodiments, the magnesium, the salt thereof, or the magnesium threonate is administered to the subject about 4 hours before sleep.

In certain embodiments, the magnesium, the salt thereof, or the magnesium threonate is administered to the subject about 4.5 hours before sleep. In further embodiments, the magnesium, the salt thereof, or the magnesium threonate is administered to the subject about 5 hours before sleep. In yet further embodiments, the magnesium, the salt thereof, or the magnesium threonate is administered to the subject about 5.5 hours before sleep. In still further embodiments, the magnesium, the salt thereof, or the magnesium threonate is administered to the subject about 6 hours before sleep.

In certain embodiments, the composition is formulated as a tablet, a gummy, a gel, a chewable tablet, a dissolvable tablet, a dissolvable sheet, a microencapsulated capsule, an elixir, a syrup, a sachet, a liquid, a mouthwash, a tincture or a paste. In further embodiments, the composition is formulated for sublingual absorption. In certain preferred embodiments, the composition is formulated for oral administration.

Oral administration can allow for the absorption of the bioactive components of the composition via the mucous membranes of the mouth or via the intestinal tract. In certain embodiments, the composition is administered orally as a gel. In further embodiments, the composition is administered as a dissolvable tablet that dissolves in the mouth. In yet further embodiments, the composition is administered as a gel like sheet that dissolves on the tongue. In still further embodiments, the composition is administered as a liquid, such as a mouthwash, ready-to-drink beverage, or a powder that can be dissolved in a liquid, such as water. In still further embodiments, the composition is administered as a tincture. In certain embodiments, the composition is formulated as a dispersible powder, a beverage, a hard capsule, or a soft capsule. In certain embodiments, the composition is formulated for extended release, controlled release, or a combination thereof. In further embodiments, the composition is formulated for sustained or prolonged release. In yet further embodiments, the composition comprises a pharmaceutically acceptable excipient.

In certain embodiments, the magnesium, the salt thereof, or the magnesium threonate is administered daily for a period of greater than 1 day. In further embodiments, the magnesium, the salt thereof, or the magnesium threonate is administered daily for a period of greater than 2 days. In yet further embodiments, the magnesium, the salt thereof, or the magnesium threonate is administered daily for a period of greater than 3 days. In still further embodiments, the magnesium, the salt thereof, or the magnesium threonate is administered daily for a period of greater than 4 days. In certain embodiments, the magnesium, the salt thereof, or the magnesium threonate is administered daily for a period of greater than 5 days. In further embodiments, the magnesium, the salt thereof, or the magnesium threonate is administered daily for a period of greater than 6 days. In yet further embodiments, the magnesium, the salt thereof, or the magnesium threonate is administered daily for a period of greater than 7 days.

In certain embodiments, the magnesium, the salt thereof, or the magnesium threonate is administered daily for a period of greater than 8 days. In further embodiments, the magnesium, the salt thereof, or the magnesium threonate is administered daily for a period of greater than 9 days. In yet further embodiments, the magnesium, the salt thereof, or the magnesium threonate is administered daily for a period of greater than 10 days. In still further embodiments, the magnesium, the salt thereof, or the magnesium threonate is administered daily for a period of greater than 11 days. In certain embodiments, the magnesium, the salt thereof, or the magnesium threonate is administered daily for a period of greater than 12 days. In further embodiments, the magnesium, the salt thereof, or the magnesium threonate is administered daily for a period of greater than 13 days.

In certain preferred embodiments, the magnesium, the salt thereof, or the magnesium threonate is administered daily for a period of greater than 14 days. In further embodiments, the magnesium, the salt thereof, or the magnesium threonate is administered daily for a period of greater than 15 days. In yet further embodiments, the magnesium, the salt thereof, or the magnesium threonate is administered daily for a period of greater than 16 days. In still further embodiments, the magnesium, the salt thereof, or the magnesium threonate is administered daily for a period of greater than 17 days. In certain embodiments, the magnesium, the salt thereof, or the magnesium threonate is administered daily for a period of greater than 18 days. In further embodiments, the magnesium, the salt thereof, or the magnesium threonate is administered daily for a period of greater than 19 days. In yet further embodiments, the magnesium, the salt thereof, or the magnesium threonate is administered daily for a period of greater than 20 days.

In certain embodiments, the magnesium, the salt thereof, or the magnesium threonate is administered daily for a period of greater than 21 days. In further embodiments, the magnesium, the salt thereof, or the magnesium threonate is administered daily for a period of greater than 22 days. In yet further embodiments, the magnesium, the salt thereof, or the magnesium threonate is administered daily for a period of greater than 23 days. In still further embodiments, the magnesium, the salt thereof, or the magnesium threonate is administered daily for a period of greater than 24 days. In certain embodiments, the magnesium, the salt thereof, or the magnesium threonate is administered daily for a period of greater than 25 days. In further embodiments, the magnesium, the salt thereof, or the magnesium threonate is administered daily for a period of greater than 26 days. In yet further embodiments, the magnesium, the salt thereof, or the magnesium threonate is administered daily for a period of greater than 27 days. In still further embodiments, the magnesium, the salt thereof, or the magnesium threonate is administered daily for a period of greater than 28 days.

In certain embodiments, the magnesium, the salt thereof, or the magnesium threonate is administered daily for a period of about 1 day. In further embodiments, the magnesium, the salt thereof, or the magnesium threonate is administered daily for a period of about 2 days. In yet further embodiments, the magnesium, the salt thereof, or the magnesium threonate is administered daily for a period of about 3 days. In still further embodiments, the magnesium, the salt thereof, or the magnesium threonate is administered daily for a period of about 4 days. In certain embodiments, the magnesium, the salt thereof, or the magnesium threonate is administered daily for a period of about 5 days. In further embodiments, the magnesium, the salt thereof, or the magnesium threonate is administered daily for a period of about 6 days. In yet further embodiments, the magnesium, the salt thereof, or the magnesium threonate is administered daily for a period of about 7 days.

In certain embodiments, the magnesium, the salt thereof, or the magnesium threonate is administered daily for a period of about 8 days. In further embodiments, the magnesium, the salt thereof, or the magnesium threonate is administered daily for a period of about 9 days. In yet further embodiments, the magnesium, the salt thereof, or the magnesium threonate is administered daily for a period of about 10 days. In still further embodiments, the magnesium, the salt thereof, or the magnesium threonate is administered daily for a period of about 11 days. In certain embodiments, the magnesium, the salt thereof, or the magnesium threonate is administered daily for a period of about 12 days. In further embodiments, the magnesium, the salt thereof, or the magnesium threonate is administered daily for a period of about 13 days.

In certain preferred embodiments, the magnesium, the salt thereof, or the magnesium threonate is administered daily for a period of about 14 days. In further embodiments, the magnesium, the salt thereof, or the magnesium threonate is administered daily for a period of about 15 days. In yet further embodiments, the magnesium, the salt thereof, or the magnesium threonate is administered daily for a period of about 16 days. In still further embodiments, the magnesium, the salt thereof, or the magnesium threonate is administered daily for a period of about 17 days. In certain embodiments, the magnesium, the salt thereof, or the magnesium threonate is administered daily for a period of about 18 days. In further embodiments, the magnesium, the salt thereof, or the magnesium threonate is administered daily for a period of about 19 days. In yet further embodiments, the magnesium, the salt thereof, or the magnesium threonate is administered daily for a period of about 20 days.

In certain embodiments, the magnesium, the salt thereof, or the magnesium threonate is administered daily for a period of about 21 days. In further embodiments, the magnesium, the salt thereof, or the magnesium threonate is administered daily for a period of about 22 days. In yet further embodiments, the magnesium, the salt thereof, or the magnesium threonate is administered daily for a period of about 23 days. In still further embodiments, the magnesium, the salt thereof, or the magnesium threonate is administered daily for a period of about 24 days. In certain embodiments, the magnesium, the salt thereof, or the magnesium threonate is administered daily for a period of about 25 days. In further embodiments, the magnesium, the salt thereof, or the magnesium threonate is administered daily for a period of about 26 days. In yet further embodiments, the magnesium, the salt thereof, or the magnesium threonate is administered daily for a period of about 27 days. In still further embodiments, the magnesium, the salt thereof, or the magnesium threonate is administered daily for a period of about 28 days.

In certain embodiments, the method further decreases nighttime stress. In certain embodiments, the method further decreases daytime dysfunction. In certain embodiments, the method further decreases the risk of developing Alzheimer's disease.

In certain embodiments, the magnesium threonate is magnesium L-threonate (Magtein®), which is a dietary supplement. In further embodiments, the magnesium threonate is magnesium L-threonate monohydrate. In yet further embodiments, the magnesium threonate is a white to off-white powder and is water soluble, forming a clear solution when the powder is dissolved in water, 1% powder at 25° C. In still further embodiments, the bulk density of the magnesium threonate is not less than about 0.4 glee (grams per cubic centimeter), particle size is not less than 90% through a US #20 and not more than 60% through a US #20, and/or loss on drying is not more than 5%.

In certain embodiments, the magnesium threonate includes about 7.2% to about 8.3% magnesium (by ICP-MS), not less than about 85% L-threonic acid (by HPLC) and/or not more than about 5000 ppm residual solvents (ethanol) (by GC-MS). In further embodiments, the magnesium threonate includes about 86% to about 91% L-threonic acid. In yet further embodiments, the magnesium threonate in aqueous solution (1% water) has a pH of 5.8-7.0, arsenic in an amount not more than about 0.5 ppm and/or cadmium in an amount of not more than about 0.2 ppm, lead in an amount of not more than about 0.5 ppm, and/or cadmium in an amount of not more than about 0.2 ppm (all except pH by ICP-MS). In still further embodiments, the magnesium threonate has a total microbiological plate count which is not more than about 3000 cfu/g, yeast and mold are not more than about 300 cfu/g and/or the magnesium threonate is negative for E. coli and Salmonella (all by USP 2021 except Salmonella, by AOAC 998.09).

In certain embodiments, the magnesium threonate does not contain animal sourced raw materials and is suitable for vegetarians and vegans. In further embodiments, the magnesium threonate is free of genetically modified organisms (GMO) and bovine spongiform encephalopathy/transmissible spongiform encephalopathy (BSE/TSE) risk. In yet further embodiments, the magnesium threonate is non-irradiated and non-ETO treated. In still further embodiments, the shelf life of the magnesium threonate is three years from the date of manufacture.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art.

Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

As used herein, the term “subject” refers to animals which can be treated using the compositions and methods of the present disclosure. Examples of animals include mammals, such as mice, rabbits, rats, horses, goats, dogs, cats, pigs, cattle, sheep, and primates (e.g. chimpanzees, gorillas, and, preferably, humans). In some embodiments, the term “subject” as used herein refers to an animal. In some embodiments, the subject is a mammal. In some embodiments, the subject is a human. In some embodiments, the subject is an adult. In certain embodiments, the adult is an individual who has reached the age of majority, for example 18 years or older. In certain embodiments, the human is female. According to one or more embodiments, the human is male.

As used herein, the term “healthy subject” refers to an animal, particularly a mammal, that does not exhibit any signs or symptoms of disease or cognitive impairment relevant to a method of administration being contemplated and is in a state of overall well-being. “Healthy subjects” are free from any acute or chronic illnesses, diseases, or medical conditions that could affect the outcome of the uses described herein.

As used herein, the term “cognitive impairment” refers to a condition in which a subject experiences a cognition impairment or deficiency. This may result from a diagnosed condition or from the subject's personal perception of impairment or deficiency.

As used herein, the term “Rapid Eye Movement” or “REM” refers to a distinct phase of sleep characterized by rapid and random movement of the eyes, low muscle tone throughout the body, and the propensity for vivid dreams. The occurrence of REM sleep can be identified through polysomnography, which measures various physiological parameters including eye movements, brain waves, and muscle activity. Improvement in REM sleep can be evaluated through specific, standardized assessment tools and questionnaires, preferably by using Oura ring measurements as disclosed herein.

As used herein, the term “deep sleep” refers to the most restorative and rejuvenating stage of sleep, characterized by slow breathing, a regular heartbeat, and relaxed muscles. During this stage, the body undergoes critical healing processes, such as cell replacement, muscle tissue building, and wound healing. Deep sleep typically encompasses between 0% and 35% of total sleep, with the average adult spending about 15-20% of their total sleep time in this stage. Deep sleep plays a vital role in thinking, creativity, and memory consolidation, particularly for procedural and declarative memory in terms of cognitive function. This stage allows the brain to rest and recover, thereby improving cognitive function, concentration, and alertness. Additionally, during deep sleep, the brain processes and integrates new information, which enhances memory retention and learning. Improvement in deep sleep can be evaluated through specific, standardized assessment tools and questionnaires, preferably by using Oura ring measurements as disclosed herein.

As used herein, the term “improves sleep” refers to the enhancement of various aspects of sleep quality and quantity, leading to a more restful and restorative sleep experience, as measured by specific, standardized assessment tools and devices. Improvement in sleep involves improving deep sleep, REM sleep, and/or overall sleep quality, which can be measured through questionnaires and sleep trackers. Improvement in sleep can be evaluated through: Insomnia Severity Index, Leeds Sleep Evaluation Questionnaire and Restorative Sleep Questionnaire, and preferably by using Oura ring measurements as disclosed herein.

As used herein, the term “improves mood” refers to the enhancement of an individual emotional state, leading to a more positive and balanced mental outlook. Improvement in mood can be evaluated through specific, standardized assessment tools and questionnaires, preferably by using Restorative Sleep Questionnaire, Profile of Mood States (POMS) Short Form and post-study questionnaire as disclosed herein.

As used herein, the term “improves morning freshness” refers to the enhancement of an individual's sense of alertness, vitality, and overall well-being upon waking after a night's sleep. Improvement in morning freshness can be evaluated through specific, standardized assessment tools and questionnaires, preferably by using the Restorative Sleep Questionnaire and other post-study questionnaires, as described herein.

In some embodiments, the compositions disclosed herein are in the form of pharmaceutically effective salts. The phrase “pharmaceutically acceptable salt(s),” is art recognized and, as used herein includes, but is not limited to, salts of acidic or basic groups that may be present in the compositions disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds present in the compositions disclosed herein that include an amino moiety also can form pharmaceutically acceptable salts with various amino acids, in addition to the acids mentioned above. Compounds present in the compositions disclosed herein that are acidic in nature are capable of forming base salts with various pharmacologically acceptable cations. Non limiting examples of such salts include alkali metal or alkaline earth metal salts and, particularly, calcium, magnesium, sodium lithium, zinc, potassium, silicon, phosphorus and iron salts. In certain embodiments, “pharmaceutically acceptable salt” or “salt” is used herein to refer to an acid addition salt or a basic addition salt which is suitable for or compatible with the treatment of patients.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients; such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In one embodiment, the pharmaceutically acceptable carrier is suitable for intravenous administration. In another embodiment, the pharmaceutically acceptable carrier is suitable for locoregional injection. In some embodiments, the phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

As described herein, the term “effective” can mean an amount that is capable of achieving the results specified in the claims and the disclosure as described herein. The term “effective” can refer to a “pharmaceutically effective amount” or a “nutraceutically effective amount” as these terms be used interchangeably as set forth herein. One of skill in the art would immediately envisage the meaning and scope of these terms depending upon their usage in the surrounding context. For further context, a pharmaceutically effective amount can often refer to an amount that is administered to achieve a therapeutic result whereas a nutraceutically effective amount can be an amount of a composition that is administered to maintain healthy levels of a particular biomarker, physical characteristic, or other health indicator. In some embodiments, an effective amount can refer to an amount that is capable of maintaining homeostatic levels of a particular characteristic of a subject or returning a subject to homeostatic levels of a particular characteristic. In some embodiments, a “therapeutically effective amount” or a “therapeutically effective dose” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation or administered using a device for such slow or extended release.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “magnesium threonate” refers to in a magnesium salt with two threonic acid anions. Magnesium threonate also may be in the form of magnesium L-threonate, magnesium (2R,3S)-2,3,4-trihydroxybutanoate, L-Threonic acid Magnesium salt (L-TAMS), or hydrates thereof.

As used herein, the term “bioavailability” refers to the rate and extent to which an agent, or the active form thereof, is absorbed from a product (e.g., an oral dosage form) and becomes available at the site of action. See U.S. Code of Federal Regulations, Title 21, Part 320.1 (2001 ed.). For oral dosage forms, bioavailability generally relates to the processes by which the agent is released from the oral dosage form (e.g., a tablet), converted to the active form (if not already in the active form), and transported either passively or actively to the site of action, e.g., absorbed into the systemic circulation. A change in bioavailability attributable to oral dosage forms may be determined by measuring total systemic drug concentrations over time after administration of different oral dosage forms. One aspect of bioavailability is the area under the curve (AUC). The AUC may be the integrated measure of systemic agent concentrations over time in units of mass-time/volume (e.g., microgram-hour/milliliter, or μg-hour/ml). Alternatively, or in addition to, the AUC may be the integrated measure of systemic agent concentrations over a defined, measurable length of time. The AUC over the first 12 or 24 hours following administration of an oral dosage form may be referred to as AUC₀₋₁₂ or AUC₀₋₂₄ respectively.

As used herein, the term “dietary supplement” according to the present disclosure refers to a composition that may be orally administered as an addition to a subject's diet, which is not a natural or conventional food, which when administered improves sleep or sleep quality in the subject, as discussed herein.

### EXAMPLES

The invention, now being generally described, it will be more readily understood by reference to the following examples, which are included merely for purposes of illustration of certain aspects and embodiments of the present invention and are not intended to limit the invention.

### Example 1: Clinical Trial Evaluating the Effects of Magnesium L-Threonate Supplementation on Sleep Parameters

The objective of the experiment was to investigate the impact of Magnesium L-threonate supplementation on sleep parameters, as well as on mood, daytime energy, and other related measurements. With this randomized placebo-controlled double-blind trial, the effectiveness of magnesium supplementation on sleep quality and daytime activity was examined, using Magnesium L-threonate in healthy adults with non-clinical insomnia (i.e., self-assessed sleep problems). Participants underwent Magnesium L-threonate supplementation for 21 days, with sleep and daytime mood and activity outcomes assessed through standardized psychometric questionnaire accompanied by objective Oura ring measurements, with the aim of discerning the effects of Magnesium L-threonate on sleep, daily functioning, and emotional states.

Primary outcomes measures were sleep quality measured weekly with the Insomnia Severity Index, Leeds Sleep Evaluation Questionnaire and Restorative Sleep Questionnaire, and the Oura ring nightly. Secondary outcome measures were a weekly mood questionnaire, daily activity measured by Oura ring, daily diary and study perceived effectiveness using a post-study questionnaire at day 21. See Hausenblas, H. A., Lynch, T., Hooper, S., Shrestha, A., Rosendale, D., & Gu, J. (2024). Magnesium L-threonate improves sleep quality and daytime functioning in adults with self-reported sleep problems: A randomized controlled trial. Sleep Medicine: X, 8, 100121, the content of which is hereby incorporated by reference in its entirety.

### Materials and Methods

### Study Design, Recruitment and Intervention

A randomized double-blind placebo-controlled, parallel-arm (1:1 allocation ratio) trial design was employed, with 80 participants. It is structured and reported according to CONSORT guidelines. The clinical trial was conducted by Wellness Discovery Labs, Jacksonville, FL, USA from Mar. 10, 2022, to Oct. 18, 2022, under the supervision of a qualified investigator (QI). This study was reviewed by the research ethics board of Sterling IRB (Protocol Number 9806, Approval Date: Mar. 10, 2022). Recruitment was for twelve weeks after that date (until June 2022). The study was closed upon receipt of the final participant's data twelve weeks after that (September 2022). The study is registered with the ISRCTN registry with study number ISRCTN14728094: https://www.isrctn.com/ISRCTN14728094.

Participants were recruited via social media announcements and from a participant recruitment list possessed by Wellness Discovery Labs. Interested participants completed a prescreen survey to determine eligibility (i.e., Insomnia Severity Index, inclusion/exclusion criteria and demographic information, e.g., age, health status, and health behaviors). Participants who met the selection criteria (based on responses to the prescreen survey) were contacted to discuss the intervention and provide institutional-review board-approved informed consent. The inclusion and exclusion criteria are described below.

Individuals included were those aged between 35 and 55 who self-reported poor sleep quality, determined by a score of between 8 and 21 on the insomnia severity index (ISI); weight between 50 and 100 kg (i.e., 110-220 lbs); and not meeting any of the exclusion criteria.

Individuals excluded were those meeting any of the following criteria: a history of sleep-affecting disorders; recent highly stressful events within 2 weeks of baseline; use of sleep supplements or medications; usage of sleep-pattern-influencing medications within 1 month of baseline; use of calcium channel blockers, anxiolytics or SSRIs, no more than 5 times per month, and not within seven days of baseline; current hormone therapy; unstable use of other medication; excessive alcohol consumption; smoking; elevated caffeine intake; irregular sleep-inducing work schedules; recent travel to different time zones within 1 month of study; pregnancy, attempts at conception, or breastfeeding; refusal to abstain from other magnesium products for two weeks before and during the trial; and individuals incompatible with the study protocol.

Consenting participants were randomly assigned participant ID numbers (using the random number function in Microsoft Excel) which assigned them into two equal groups, a 1:1 allocation ratio). These groups were either the Magnesium L-threonate group or placebo group. Participants were sent an investigational product or placebo, with usage instructions. The placebo was rice protein powder. Magnesium L-threonate and placebo were identical in taste and appearance, in identical capsules, in identical bottles separated by lot numbers. Participants were blind to treatment. Investigators remained blind to treatment: lot number contents were only revealed after statistical analysis. As a decentralized trial, participants did not visit a clinic, all recruitments, contact, screening, consenting, and assessments were performed online. Participant flow is shown in FIG. 1.

The Magnesium L-threonate supplement used, Magtein®, is a brain-bioavailable magnesium L-threonate, produced and supplied by Threotech LLC (NV, USA). Magtein® contains about 75 mg/g of elemental magnesium. Participants were directed to consume 1 g/d of the testing products: 2 capsules each containing either 500 mg Magnesium L-threonate or identically appearing placebo (rice protein powder, AIDP Inc., CA, USA) 2 h before bedtime.

### Outcome Measures

Primary outcomes measures were sleep quality measured weekly with the Insomnia Severity Index, Leeds Sleep Evaluation Questionnaire and Restorative Sleep Questionnaire, and the Oura ring nightly. Secondary outcome measures were a weekly mood questionnaire, daily activity measured by Oura ring, daily diary and study perceived effectiveness using a post-study questionnaire at day 21.

Participants were to complete psychometric self-report questionnaires on day 0 (baseline), day 7, day 14, and day 21. In addition, participants were to maintain a daily diary to document subjective sleep aspects, adherence, and adverse events. Participants also wore an Oura Ring to objectively determine sleep and daytime activity. Participants maintained their current lifestyle behaviors and did not engage in any new forms of structured exercise or begin a new diet or health intervention during the trial. At the end of the study, participants completed a post-study questionnaire.

Participants completed the Insomnia Severity Index (ISI), a self-report measure assessing symptoms of recent insomnia. This index assesses sleep onset, sleep maintenance, early morning awakening problems; sleep dissatisfaction; interference of sleep difficulties with daytime functioning; whether sleep problems are noticed by others; and distress caused by sleep difficulties. A 5-point Likert scale is used to rate each item (e.g., 0=no problem; 4=very severe problem), yielding a total score ranging from 0 to 28. The total score is interpreted as follows: absence of insomnia (0-7); sub-threshold insomnia (8-14); moderate insomnia symptoms (15-21); and severe insomnia symptoms (22-28). The Insomnia Severity Index (ISI) has good internal consistency (Cronbach alpha=0.91). This was assessed at recruitment, and weekly throughout the study.

Participants completed the Leeds Sleep Evaluation Questionnaire (LSEQ), comprising self-rating questions concerned with aspects of sleep and early morning behavior. The questionnaire has been used to monitor subjectively perceived changes in sleep in a variety of populations. The questionnaire was reworded from “medication” to “supplementation” in this study. This scale has good psychometric properties. Scoring categories reported here are behavior following awakening, awake following sleep, quality of sleep and getting to sleep. Higher scores indicate an improvement in sleep quality. This was assessed weekly.

Participants completed the Restorative Sleep Questionnaire (RSQ), which measures refreshing quality of sleep. Nonrestorative sleep is one of the cardinal symptoms of insomnia and can occur independent of other components of insomnia. This questionnaire used nine questions with answers scaled from 1 to 5: grouchy, in a good mood, mentally alert, sleepy, tired, rested, refreshed and restored, ready to start the day, and energetic. The total score is an average score based on all nine items. This questionnaire has good psychometric properties. Higher scores indicate an improvement in sleep quality, except noted otherwise. This was assessed weekly.

Participants completed the Profile of Mood States (POMS) short form to assess mood states: it includes scores for tension, anger, vigor, fatigue, depression, esteem, and confusion. A composite total score was computed by summing each of the individual scores for tension, depression, anxiety, fatigue, and confusion, with vigor and esteem scores subtracted to indicate participants' total mood disturbance. Each item of the POMS short form was scored on a 5-point Likert scale ranging from 0 (not at all) to 4 (extremely). This scale has good-to-excellent reliability and validity. This was assessed weekly.

Participants completed daily diaries encompassing aspects of day-time energy/productivity, events affecting sleep quality, recorded adverse events and adherence.

To objectively measure sleep quality and daytime functioning the Oura ring was employed, a waterproof wearable multisensory device that quantifies daily physical activity, night-time sleep duration, and estimates sleep stages (https://ouraring.com/). The ring also measures motion and body temperature. According to the manufacturer, the ring is worn on the index finger and uses physiological signals combination of motion, heart rate, heart rate variability, and pulse wave variability amplitude) in combination with machine learning-based methods to calculate deep, light and REM sleep in addition to sleep/wake states. The Oura ring has high validity in the assessment of nocturnal heart rate, heart rate variability, movement, and sleep out-comes in healthy adults in natural environment.

The Oura ring measurements come in three main categories: Sleep, Activity and Readiness. In the Sleep category, there are 15 measurements: (1) sleep duration, (2) time awake, (3) light sleep, (4) sleep latency (how quickly one fell asleep), (5) REM sleep, (6) deep sleep, (7) total sleep; (8) sleep efficiency (how much time in bed was actually spent sleeping) (these scores 1-8 measured in minutes); (9) overall sleep score, (10) deep sleep score, (11) sleep disturbance score, (12) sleep efficiency score, (13) latency sleep score, (14) REM sleep score, (15) total sleep score (these scores 9-15 calculated using manufacturer proprietary algorithms by combining the resting heart rate, body temperature, movement, and time spent in specific sleep stages); In the Activity category, there are three measurements: (1) activity score, (2) activity met daily movement goals, (3) activity daily movement score; In the Readiness category, there are three measurements: (1) readiness score, (2) readiness activity balance, and (3) readiness sleep balance. Activity scores are measured through daily outputs (e.g., step counts, training frequencies etc.) using a built-in accelerometer. Readiness scores comprise eight contributors (body temperature, sleep, heart rate variability, sleep balance, previous day activity, activity balance, resting heart rate, and recovery index) over 14-day weighted averages (with the most recent 2-5 days weighted more highly) that provide an indication of daily activity. Readiness activity balance compares recent with longer term average activity to estimate acclimatization to the demands of activity. These scores are calculated using manufacturer proprietary algorithms. Data from weekly averages were used.

Participants completed post-study questionnaires at the end of the study. These were yes/no queries regarding whether taking the product had a positive effect on their perceived stress, anxiety, or mood, as follows:

Please circle Yes or No to the below questions based on your experience over the past month. After taking the supplement:
Has the quality of your sleep improved. Yes/No
Is your sleep more refreshing/restoring? Yes/No
Do you feel more rested upon awakening? Yes/No
Do you feel that your physical health has improved? Yes/No
Do you feel that your mental health has improved? Yes/No
Do you feel calmer? Yes/No
Do you feel that your memory during the daytime improved? Yes/No
Do you feel that your concentration during the daytime improved? Yes/No
Do you feel like you have more energy during the daytime? Yes/No Do you feel more alert during the daytime? Yes/No

In addition, the participants were also asked to complete the following two questions using a Likert scale anchored with 1 (strongly agree) to 5 (strongly disagree):
The supplement had a positive effect on my perceived stress and anxiety.
The supplement had a positive effect on my mood.

### Statistical Analysis

Determination of Sample Size. A sample size of 80 subjects (40 per arm) was considered sufficient to detect a significant difference between groups with 80% power and a 5% level of significance, allowing for 20% drop out rate.

The intent to treat (ITT) population were analyzed. Data were analyzed for normality using Shapiro-Wilk test and Q-Q plot. Outliers were characterized as data points that exceeded three interquartile ranges beyond 25th and 75th percentiles. However, no extreme outliers were observed. Continuous data are presented as Mean and standard deviation (SD) and analyzed using linear mixed model with condition, and time as fixed factors and subject as random factor. Multiple comparisons were corrected using Sidak Adjustment. Categorical variables were analyzed using Chisquare test and expressed as counts/percentage where appropriate. Statistical analyses were performed using Statistical Product and Service Solutions (SPSS) version 27. Post hoc analyses (T-tests) were performed using R (version 4.2.2).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for improving sleep in a human subject in need thereof, comprising administering to the subject an effective amount of magnesium or a salt thereof, wherein the effective amount is from about 1 mg to about 300 mg based on the weight of magnesium.

2. The method of claim 1, wherein the effective amount of the magnesium or salt thereof is from about 72 mg to about 83 mg based on the weight of magnesium.

3. (canceled)

4. The method of claim 1, wherein the method comprises administering a magnesium salt, wherein the effective amount is an amount which is sufficient to deliver from about 72 mg to about 83 mg of magnesium to the subject per gram of the magnesium salt.

5. (canceled)

6. The method of claim 4, wherein the magnesium salt is magnesium threonate.

7. The method of claim 6, wherein the effective amount of magnesium threonate is from about 100 mg to about 5 g.

8. A method for improving sleep in a subject in need thereof, comprising administering to the subject an effective amount of magnesium threonate.

9. A method for improving sleep quality in a human subject in need thereof, comprising administering to the subject an effective amount of magnesium threonate.

10. The method of claim 8, wherein the sleep is deep sleep.

11. The method of claim 8, wherein the sleep is Rapid Eye Movement (REM) sleep.

12. A method for improving mood in a human subject in need thereof, comprising administering to the subject an effective amount of magnesium threonate.

13. A method for improving energy and alertness in a human subject in need thereof, comprising administering to the subject in need an effective amount of magnesium threonate.

14. A method for improving daily productivity in a human subject in need thereof, wherein the method comprising administering to the subject in need an effective amount of magnesium threonate.

15. A method for improving morning freshness in a human subject in need thereof, wherein the method comprising administering to the subject in need an effective amount of magnesium threonate.

16. The method of claim 8, wherein the effective amount of magnesium threonate is from about 50 mg to about 5 g.

17-28. (canceled)

29. The method of claim 8, wherein the magnesium threonate is magnesium L-threonate.

30-32. (canceled)

33. The method of claim 8, wherein the magnesium threonate is administered orally.

34-38. (canceled)

39. The method of claim 8, wherein the method further decreases nighttime stress.

40. The method of claim 8, wherein the method further decreases daytime dysfunction.

41. The method of claim 8, wherein the method further decreases the risk of developing Alzheimer's disease.

42. The method of claim 9, wherein the effective amount of magnesium threonate is from about 50 mg to about 5 g.

43. The method of claim 9, wherein the magnesium threonate is magnesium L-threonate.
